(19) United States
(12) Patent Application Publication
(10) Pub. No.: US 2022/0266238 A1
(43) Pub. Date: Aug. 25, 2022

(54) METALLOSILICATE CATALYST REGENERATION

(71) Applicant: Dow Global Technologies LLC, Midland, MI (US)

(72) Inventors: Wen -Sheng Lee, Midland, MI (US); Mingzhe Yu, Sugar Land, TX (US); Thomas H. Peterson, Midland, MI (US); Sung-Yu Ku, Manvel, TX (US); Wanglin Yu, Pearland, TX (US); Le Wang, Pearland, TX (US); Stephen W. King, Braselton, GA (US)

(21) Appl. No.: 17/625,173

(22) PCT Filed: Sep. 29, 2020
(86) PCT No.: PCT/US2020/053200

§ 371 Date: Jan. 6, 2022

Related U.S. Application Data
(60) Provisional application No. 62/908,123, filed on Sep. 30, 2019.

(51) Int. Cl. B01J 38/02; B01J 29/70; B01J 29/90; C07C 41/06

(52) U.S. Cl. CPC B01J 38/02; B01J 29/7007; B01J 29/90; C07C 41/06

(57) ABSTRACT

According to a least one feature of the present disclosure, a method includes the steps: (a) providing a metallosilicate catalyst that has been used to catalyze a chemical reaction; and (b) heating the metallosilicate catalyst to a temperature from 200° C. to 425° C. for a period of 0.5 hours to 5 hours.

### BACKGROUND

### Field of the Invention

The present disclosure generally relates to metallosilicate catalysts and more specifically to the regeneration of metallosilicate catalysts.

### Introduction

Production of secondary alcohol ethoxylate surfactants can be carried out by the catalyzed ethoxylation of (poly)alkylene glycol monoalkyl ether (“monoalkyl ether”). The monoalkyl ether is formed from an olefin and a (poly)alkylene glycol using crystalline metallosilicate catalysts (“metallosilicate catalysts”). Metallosilicate catalysts offer a selectivity for monoalkyl ether of greater than 80% at an olefin conversion of greater than 5% which is advantageous as (poly)alkylene glycol dialkyl ether (“dialkyl ether”) are deleterious to properties of the secondary alcohol ethoxylate surfactants.

Although providing greater than 80% selectivity for monoalkyl ether, the metallosilicate catalysts foul quickly resulting in short in-service times, low monoalkyl ether production rate and the need for repeated regeneration steps for the metallosilicate catalysts. Regeneration of the metallosilicate catalysts is carried out at high temperatures for extended periods to remove the fouling agents. For example, U.S. Pat. No. 6,417,408 explains that it is preferable that the regeneration of the catalyst is carried out by calcining the catalyst at 450° C. or greater because temperatures below 450° C. were believed to leave too much residual carbon (as evidenced by the visual remainder of residual carbon) and as a result exhibit shorter time periods until the catalyst must be regenerated and a lower monoalkyl ether. The necessary repetition of the conventional regeneration process is expensive and requires specialty equipment.

Accordingly, it would be surprising to discover a metallosilicate catalyst regeneration process which is carried out at temperature below 450° C. and results in a catalyst with comparable monoalkyl ether production rates to a fresh regenerated catalyst and greater than 80% monoalkyl ether selectivity.

### SUMMARY

The present invention offers a solution to providing a catalyst regeneration process which is carried out at temperature below 450° C. and results in a catalyst with comparable monoalkyl ether production rate to a fresh regenerated catalyst and greater than 80% monoalkyl ether selectivity.

The present invention is a result of discovering that regenerating a fouled metallosilicate catalyst at a temperature of from 200° C. to 425° C. unexpectedly provides the regenerated catalyst with comparable and/or even superior monoalkyl ether production rates to a fresh regenerated catalyst and greater than 80% monoalkyl ether selectivity at olefin conversions of 5% or greater. Such a result is surprising in that regeneration temperatures of 100° C. and more below the lowest acceptable limit established by the prior art can provide monoalkyl ether production rates and selectivity values superior to higher temperature conventional processes. Even more surprising is that although conventional regeneration processes rely on oxidation of fouling, the present invention may utilize inert atmospheres or even vacuums and still achieve superior results to the conventional processes.

Accordingly, not only can energy cost savings be realized through the surprising lower temperature regeneration process, superior production rates and monoalkyl ether selectivity may also be achieved through use of the present invention.

According to at least one feature of the present disclosure, a method comprises the steps: (a) providing a metallosilicate catalyst that has been used to catalyze a chemical reaction; and (b) heating the metallosilicate catalyst to a temperature from 200° C. to 425° C. for a period of 0.5 hours to 5 hours.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standards.

IUPAC codes describing Crystal structures as delineated by the Structure Commission of the International Zeolite Association refer to the most recent designation as of the priority date of this document unless otherwise indicated.

As used herein, the term weight percent (“wt %”) designates the percentage by weight a component is of a total weight of an indicated composition.

Method

The method of the present invention is directed to the regeneration of metallosilicate catalysts. The method may comprise steps of providing a metallosilicate catalyst that has been used to catalyze a chemical reaction; and heating the metallosilicate catalyst to a temperature from 200° C. to 425° C. for a period of 0.5 hours to 5 hours. The method may further comprise steps of catalyzing the chemical reaction between an olefin and an alcohol using the metallosilicate catalyst and generating an alkylene glycol monoalkyl ether.

Olefin

The olefin used in the method may be linear, branched, acyclic, cyclic, or mixtures thereof. The olefin may have from 5 carbons to 30 carbons (i.e., C₅-C₃₀). The olefin may have 5 carbons or greater, or 6 carbons or greater, or 7 carbons or greater, or 8 carbons or greater, or 9 carbons or greater, or 10 carbons or greater, or 11 carbons or greater, or 12 carbons or greater, or 13 carbons or greater, or 14 carbons or greater, or 15 carbons or greater, or 16 carbons or greater, or 17 carbons or greater, or 18 carbons or greater, or 19 carbons or greater, or 20 carbons or greater, or 21 carbons or greater, or 22 carbons or greater, or 23 carbons or greater, or 24 carbons or greater, or 25 carbons or greater, or 26 carbons or greater, or 27 carbons or greater, or 28 carbons or greater, or 29 carbons or greater, while at the same time, 30 carbons or less, or 29 carbons or less, or 28 carbons or less, or 27 carbons or less, or 26 carbons or less, or 25 carbons or less, or 24 carbons or less, or 23 carbons or less, or 22 carbons or less, or 21 carbons or less, or 20 carbons or less, or 19 carbons or less, or 18 carbons or less, or 17 carbons or less, or 16 carbons or less, or 15 carbons or less, or 14 carbons or less, or 13 carbons or less, or 12 carbons or less, or 11 carbons or less, or 10 carbons or less, or 9 carbons or less, or 8 carbons or less, or 7 carbons or less, or 6 carbons or less.

The olefin may include alkenes such as alpha (α) olefins, internal disubstituted olefins, or cyclic structures (e.g., C₃-C₁₂ cycloalkene). α olefins include an unsaturated bond in the

α-position of the olefin. Suitable α olefins may be selected from the group consisting of propylene, 1-butene, 1-hexene, 4-methyl-1-pentene, 1-heptene, 1-octene, 1-decene, 1-dodecene, 1-tetradecene, 1-hexadecene, 1-octadecene, 1-icosene, 1-docosene and combinations thereof. Internal disubstituted olefins include an unsaturated bond not in a terminal location on the olefin. Internal olefins may be selected from the group consisting of 2-butene, 2-pentene, 2-hexene, 3-hexene, 2-heptene, 3-heptene, 2-octene, 3-octene, 4-octene, 2-nonene, 3-nonene, 4-nonene, 2-decene, 3-decene, 4-decene, 5-decene and combinations thereof. Other exemplary olefins may include butadiene and styrene.

Examples of suitable commercially available olefins include NEODENE™ 6-XHP, NEODENE™ 8, NEODENE™ 10, NEODENE™ 12, NEODENE™ 14, NEODENE™ 16, NEODENE™ 1214, NEODENE™ 1416, NEODENE™ 16148 from Shell, The Hague, Netherlands.

Alcohol

The alcohol utilized in the method may comprise a single hydroxyl group, may comprise two hydroxyl groups (i.e., a glycol) or may comprise three hydroxyl groups. The alcohol may include 1 carbon or greater, or 2 carbons or greater, or 3 carbons or greater, or 4 carbons or greater, or 5 carbons or greater, or 6 carbons or greater, or 7 carbons or greater, or 8 carbons or greater, or 9 carbons or greater, while at the same time, 10 carbons or less, or 9 carbons or less, or 8 carbons or less, or 7 carbons or less, or 6 carbons or less, or 5 carbons or less, or 4 carbons or less, or 3 carbons or less, or 2 carbons or less. The alcohol may be selected from the group consisting of methanol, ethanol, monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof. According to various examples, the alcohol is a (poly)alkylene glycol such as monoethylene glycol, diethylene glycol, propylene glycol and triethylene glycol.

A molar ratio of alcohol to olefin in the method may be from be 20:1 or less, or 15:1 or less, or 10:1 or less, or 9:1 or less, or 8:1 or less, or 7:1 or less, or 6:1 or less, or 5:1 or less, or 4:1 or less, or 3:1 or less, or 2:1 or less, or 0.2:1 or less, while at the same time, 0.1:1 or greater, or 1:1 or greater, or 1:2 or greater, or 1:3 or greater, or 1:4 or greater, or 1:5 or greater, or 1:6 or greater, or 1:7 or greater, or 1:8 or greater, or 1:9 or greater, or 1:10 or greater, or 1:15 or greater, or 1:20 or greater.

Metallosilicate Catalyst

As used herein the term “metallosilicate catalyst” is an aluminosilicate (commonly referred to as a zeolite) compound having a crystal lattice that has had one or more metal elements substituted in the crystal lattice for a silicon atom. The crystal lattice of the metallosilicate catalyst form cavities and channels inside where cations, water and/or small molecules may reside. The substitute metal element may include one or more metals selected from the group consisting of B, Al, Ga, In, Ge, Sn, P, As, Sb, Sc, Y, La, Ti, Zr, V, Cr, Mn, Pb, Pd, Pt, Au, Fe, Co, Ni, Cu, Zn. The metallosilicate catalyst may be substantially free of Hf. According to various examples, the metallosilicate may have a silica to alumina ratio of from 5:1 to 1,500:1 as measured using Neutron Activation Analysis. The silica to alumina ratio may be from 5:1 to 1,500:1, or from 10:1 to 500:1, or from 10:1 to 400:1, or from 10:1 to 300:1 or from 10:1 to 200:1. Such a silica to alumina ratio may be advantageous in providing a highly homogenous metallosilicate catalyst with an organophilic-hydrophobic selectivity that adsorb non-polar organic molecules.

The metallosilicate catalyst may have one or more ion-exchangeable cations outside the crystal lattice. The ion-exchangeable cation may include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, R₄P⁺ (where R is H or alkyl).

The metallosilicate catalyst may take a variety of crystal structures. Specific examples of the metallosilicate catalyst structures include MFI (e.g. ZSM-5), MEL (e.g. ZSM-11), BEA (e.g. β-type zeolite), FAU (e.g. Y-type zeolite), MOR (e.g. Mordenite), MTW (e.g. ZSM-12), and LTL (e.g. Linde L), as described using IUPAC codes in accordance with nomenclature by the Structure Commission of the International Zeolite Association.

The crystalline frameworks of metallosilicate catalyst are represented by networks of molecular-sized channels and cages comprised of corner-shared tetrahedral [TO₄] (T=Si or Al) primary building blocks. A negative charge can be introduced onto the framework via the isomorphous substitution of a framework tetravalent silicon by a trivalent metal (e.g., aluminum) atom. The overall charge neutrality is then achieved by the introduction of cationic species compensating for the resulting negative lattice charge. When such a charge-compensation is provided by protons, Br∅nsted acid sites are formed rendering the resulting H-forms of zeolites strong solid Br∅nsted acids.

The metallosilicate catalysts may be used in the method in a variety of forms. For example, the metallosilicate catalysts may be powdered (e.g., particles having a longest linear dimension of less than 100 micrometers), granular (e.g., particles having a longest linear dimension of 100 micrometers or greater), or molded articles of powdered and/or granular metallosilicate catalysts.

The metallosilicate catalysts may have a surface area of 100 m²/g or greater, or 200 m²/g or greater, or 300 m²/g or greater, or 400 m²/g or greater, or 500 m²/g or greater, or 600 m²/g or greater, or 700 m²/g or greater, or 800 m²/g or greater, or 900 m²/g or greater, while at the same time, 1000 m²/g or less, or 900 m²/g or less, or 800 m²/g or less, or 700 m²/g or less, or 600 m²/g or less, or 500 m²/g or less, or 400 m²/g or less, or 300 m²/g or less, or 200 m²/g or less. Surface area is measured according to ASTM D4365-19.

Metallosilicate catalysts can be synthesized by hydrothermal synthesis methods. For example, the metallosilicate catalysts can be synthesized from heating a composition comprising a silica source (e.g., silica sol, silica gel, and alkoxysilanes), a metal source (e.g., metal sulfates, metal oxides, metal halides, etc.), and a quaternary ammonium salt such as a tetraethylammonium salt or tetrapropylammonium to a temperature of about 100° C. to about 175° C. until a crystal solid forms. The resulting crystal solid is then filtered off, washed with water, and dried, and then calcined at a temperature form 350° C. to 600° C.

Examples of suitable commercially available, metallosilicate catalysts include CP814E, CP814C, CP811C-300, CBV 712, CBV 720, CBV 760, CBV 2314, CBV 10A from ZEOLYST INTERNATIONAL of Conshohocken, PA.

Generating Monoalkyl Ether

Catalyzing the chemical reaction between an olefin and an alcohol using the metallosilicate catalyst results in the generation of an alkylene glycol monoalkyl ether. The solvent is used in facilitated the chemical reaction. The chemical reaction between the olefin and the alcohol is catalyzed by the metallosilicate catalyst in a reactor to generate the monoalkyl ether. Various monoalkyl ethers may be produced for different applications by varying which olefin is utilized and/or by varying which alcohol is utilized. Monoalkyl ether are utilized for a number of applications such as solvents, surfactants, and chemical intermediates, for instance.

The reaction of the olefin and the alcohol may take place at from 50° C. to 300° C. or from 100° C. to 200° C. In a specific example the reaction may be carried out at 150° C. Reaction of the olefin and the alcohol may be carried out in a batch reactor, continuous stirred tank reactor, in a continuous fixed-bed reactor or a fluidized bed reactor. In operation of the chemical reaction, the Br∅nsted acid sites of the metallosilicate catalyst may catalyze the etherification of the olefin to the alcohol through an addition type reaction. The reaction of the olefin and the alcohol produces the monoalkyl ether.

The addition reaction of the olefin to the glycol may form not only monoalkyl ether but also the dialkyl ether. The metallosilicate catalyst may exhibit a selectivity to produce alkylene monoalkyl ether, but not dialkyl ether. The monoalkyl ether selectivity may be 70% or greater, or 75% or greater, or 80% or greater, or 85% or greater, or 90% or greater, or 95% or greater or 99% or greater, while at the same time, 100% or less, or 95% or less, or 90% or less, or 85% or less, or 80% or less, or 75% or less. The dialkyl ether selectivity may be 0% or greater, or 2% or greater, or 4% or greater, or 6% or greater, or 8% or greater, or 10% or greater, or 12% or greater, or 14% or greater, or 16% or greater, or 18% or greater, while at the same time, 20% or less, or 18% or less, or 16% or less, or 14% or less, or 12% or less, or 10% or less, or 8% or less, or 6% or less, or 4% or less, or 2% or less.

A monoalkyl ether yield is calculated by multiplying the amount of olefin conversion by the monoalkyl ether selectivity. The alkylene glycol monoalkyl ether yield may be 10% or greater, or 15% or greater, or 20% or greater, or 25% or greater, or 30% or greater, or 35% or greater, while at the same time, 40% or less, or 35% or less, or 30% or less, or 25% or less, or 20% or less, or 15% or less. Monoalkyl ether yield is a measure of the catalytic activity and selectivity and is a good measure of the production rate of the metallosilicate catalyst.

During the reaction of the olefin and the alcohol, the catalyst becomes fouled. The fouling has the effect of deactivating (i.e., lost etherification activity >50%) the catalyst within hours.

Heating the Metallosilicate Catalyst

Regeneration of the metallosilicate catalyst is performed by heating the metallosilicate catalyst to a temperature of from 200° C. to 450° C. for a period of 0.5 hours to 5 hours. Heating of the metallosilicate catalyst may be carried out in a variety of ovens, furnaces and enclosures. For example, regeneration may take place in rotary kilns, box furnaces, fluidized bed furnaces, roller-hearth kilns, enclosures such as tubes comprising a heating element and mesh belt furnaces. The metallosilicate catalyst may be removed from the reactor prior to heating and regeneration or the metallosilicate catalyst may remain in the reactor. The regeneration and heating of the metallosilicate catalyst may be performed in the absence of liquids (i.e., the metallosilicate catalyst is dried before and/or during the regeneration). For example, the metallosilicate catalyst may be removed and dried or may be dried within the reactor (e.g., for fluidized bed furnaces).

The regeneration of the metallosilicate catalyst may be performed in atmospheric oxygen (i.e., calcination), under an atmosphere which is inert to the catalyst and fouling on the metallosilicate catalyst or under a vacuum. Inert atmospheres may comprise, nitrogen, argon, helium, CO2, other gases inert to the fouling and/or combinations thereof. Inert atmospheres may comprise the inert component at 60 volume percent (“vol%”) or greater, or 70 vol% or greater, or 80 vol% or greater, or 90 vol% or greater, while at the same time, 100 vol% or less, or 90 vol% or less, or 80 vol% or less, or 70 vol% or less. Volume percent is measured at the regeneration temperature as the percent of volume occupied by inert component divided by the total cavity space that the metallosilicate catalyst is in. Such inert atmospheres may be achieved by passing the inert gas over the metallosilicate catalyst at a constant rate during the heating. The heating of the metallosilicate catalyst may be carried out under a pressure of 4000 Pa or less, or 3000 Pa or less, or 2000 Pa or less, or 1000 Pa or less, or 900 Pa or less, or 800 Pa or less, or 700 Pa or less, or 600 Pa or less, or 500 Pa or less, or 400 Pa or less, 300 Pa or less, or 200 Pa or less, or 100 Pa or less, or 50 Pa or less, or 10 Pa or less or 5 Pa or less.

The regeneration of the metallosilicate catalyst may be carried out at temperature of 200° C. or greater, or 225° C. or greater, or 250° C. or greater, or 275° C. or greater, or 300° C. or greater, or 325° C. or greater, or 350° C. or greater, or 375° C. or greater, 400° C. or greater, or 425° C. or greater, while at the same time, 450° C. or less, or 425° C. or less, or 400° C. or less, or 375° C. or less, or 350° C. or less, or 325° C. or less, or 300° C. or less, or 275° C. or less, or 250° C. or less, or 225° C. or less.

The regeneration of the metallosilicate catalyst may be carried out for a time period of 0.5 hours or greater, or 0.75 hours or greater, or 1.00 hours or greater, or 1.25 hours or greater, or 1.50 hours or greater, or 1.75 hours or greater, or 2.00 hours or greater, or 2.25 hours or greater, or 2.50 hours or greater, or 2.75 hours or greater, or 3.00 hours or greater, or 3.25 hours or greater, or 3.50 hours or greater, or 3.75 hours or greater, or 4.00 hours or greater, or 4.25 hours or greater, or 4.50 hours or greater, or 4.75 hours or greater, while at the same time, 5.00 hours or less, or 4.75 hours or less, or 4.50 hours or less, or 4.25 hours or less, or 4.00 hours or less, or 3.75 hours or less, or 3.50 hours or less, or 3.25 hours or less, or 3.00 hours or less, or 2.75 hours or less, or 2.50 hours or less, or 2.25 hours or less, or 2.00 hours or less, or 1.75 hours or less, or 1.50 hours or less, or 1.25 hours or less, or 1.00 hours or less, or 0.75 hours or less.

Advantages

Use of the present invention may offer a variety of advantages. First, cost savings related to energy usage may be achieved. Conventional regeneration of catalysts often require heat in excess of 450° C. for multiple hours which is expensive. Use of temperatures between 200° C. and 425° C. reduces the energy burden of regenerating the catalyst and thus decreases overall production costs. Second, higher production rates of monoalkyl ether by the catalyst may result in greater yields of monoalkyl ether for the same given time interval. Conventional regeneration of catalysts at best recovered catalyst activity to fresh catalyst levels. Use of temperatures between 200° C. and 425° C. to regenerate catalysts may offer greater catalytic activity to regenerated catalysts than fresh catalysts exhibit. Third, the variety of heating environments (e.g., air, inert and/or vacuum) offers process flexibility.

### EXAMPLES

Materials

Catalyst is a metallosilicate catalysts defined by a BEA structure and having a silica to alumina ratio of 25:1 and a surface area of 680 m²/g, that is commercially available as CP814E from ZEOLYST INTERNATIONAL™ of Conshohocken, PA.

Olefin is 1-Dodecene alpha olefin that is commercially available as NEODENE™ 12 from the SHELLυ group of The Hague, Netherlands.

Monoethylene Glycol is liquid anhydrous ethylene glycol purchased from SIGMA ALDRICH™ having a CAS Number of 107-21-1.

Test Methods

Gas Chromatography Samples

Prepare gas chromatography samples by mixing 100 μL of the example with 10 mL of gas chromatography solution that was prepared by addition of 1 mL of hexadecane in 1 L of ethyl acetate. Analyze the sample using an Agilent 7890B gas chromatography instrument. Determine the total amount of 1-dodecene derived species, which includes monoalkyl ether, dialkyl ether and 2-dodecanol, total amount of dodecene, which includes 1-dodecene and all non 1-dodecene other C₁₂ isomers. Table 1 provides the relevant gas chromatography instrument parameters.

TABLE 1

| | |
|---|---|
| Chromatograph: | Agilent 7890 Series GC |
| Column: | Agilent HP88, 100 m × 0.25 mm × 0.20 um |
| Detector | FID |
| Oven: | 50° C. - 7 min - 6° C./min - 260° C. - 1 min |
| Injector: | 250° C. |
| Detector: | 300° C. |
| Carrier: | Helium 2.0 mL/min constant flow mode |
| Split ratio: | 10 |
| Make-Up: | Nitrogen 25 mL/min |
| Air: | 400 mL/min |
| Hydrogen: | 40 mL/min |
| Inlet Liner: | Restek PN 23305.5 Sky Precision |
| | Liner with wool |
| Sample Size: | 1 μL |
| GC vial rinsing solvent: | ethyl acetate |

Olefin Conversion

Calculate the percent olefin conversion by dividing the total amount of dodecene derived species by the summation of total amount of dodecene derived species and the amount of dodecene. Multiply the quotient by 100.

Monoalkyl Ether Selectivity

Calculate the percent monoalkyl ether selectivity by dividing the total amount of monoalkyl ether by the total amount of dodecene derived species. Multiply the quotient by 100.

Monoalkyl Ether Yield

Calculate the monoalkyl ether yield by multiplying the olefin conversion value by the monoalkyl ether selectivity value.

Normalized Yield

Calculate normalized yield by dividing the monoalkyl ether yield by catalyst loading.

Sample Preparation

Spent Air Catalyst

Load 67g of monoethylene glycol, 62g of olefin and 7.5 g of catalyst into a 300 mL Parr reactor with a heating jacket and controller to form a reaction mixture. Sealed the reactor and heat to 150° C. under 1100 rotations per minute (rpm) agitation using a pitch blade impeller. Allow for 1 hour of reaction. Remove the reaction mixture from the reactor and isolate the catalyst via centrifugation. Repeat four times to collect sufficient spent catalyst. Transfer the spent catalyst to four ceramic dishes and dry the spent catalyst in a box oven with constant air flow at 110° C. for 12 hours. Grind the spent catalyst into powder using a mortar and pestle and mix the spent catalyst in a bottle to create a single source of dried, spent catalyst.

Spent Vacuum and Nitrogen Catalyst

Load 67 g of monoethylene glycol, 62 g of olefin and 7.5 g of catalyst into a 300 mL Parr reactor with a heating jacket and controller to form a reaction mixture. Sealed the reactor and heat to 150° C. under 1100 rpm agitation using a pitch blade impeller. Allow for 3.5 hours of reaction. Remove the reaction mixture from the reactor and isolate the catalyst via centrifugation. Repeat four times to collect sufficient spent catalyst. Transfer the spent catalyst to four ceramic dishes and dry the spent catalyst in a box oven with constant air flow at 105° C. for 8 hours. Grind the spent catalyst into powder using a mortar and pestle and mix the spent catalyst in a bottle to create a single source of dried, spent catalyst.

Fresh Catalyst preparation

Place a portion of the catalyst fresh from the vendor on a ceramic dish and calcine in a box oven with constant air flow at a temperature of 550° C. for 12 hours.

Air Regenerated Catalysts

Place a portion of the dried spent catalysts on a ceramic dish and calcine in a box oven with constant air flow at the designated temperature for the designated time.

Nitrogen Regenerated Catalysts

Place a portion of the dried spent catalysts on a ceramic dish and place in a box oven with a constant flow of nitrogen (N₂) at the designated temperature for the designated time.

Vacuum Regenerated Catalysts

Place a portion of the dried spent catalysts in a glass tube having an open end and a closed end. Connect a vacuum pump to the open end of the tube and place a heating jacket around the tube. Remove air present in the tube until a pressure of 6.65 Pa (50 μm of mercury) is reached and heat the sample at the designated temperature for the designated time.

Results

Test catalytic activity of samples by placing 6.2 g of 1-dodecene and 6.7 g of monoethylene glycol in a 40 mL vial reactor with a rare earth magnetic stir bar. Set the magnetic stir bar to stir in a tumbling style. Heat the vial reactor contents to a reaction temperature of 150° C. Comparative example (“CE”) CE1-CE4 and inventive examples (“IE”) IE1-IE13 were all reacted for 1 hour while CE5-CE6 and 1E14-1E16 were reacted for 1.5 hours. CE1, CE3 and CE5 are fresh catalyst samples and CE2, CE4 and CE6 are the corresponding spent (i.e., unregenerated) CE1, CE3, and CE5 respectively.

Table 2 provides catalytic performance for a variety of catalyst regeneration conditions.

TABLE 2

| | | Catalyst | Olefin | Monoalkyl | Monoalkyl | Normalized |
|---|---|---|---|---|---|---|
| | Regeneration | Loading | Conversion. | Ether | Ether yield | Yield |
| Ex. | conditions | (g) | (%) | selectivity (%) | (%) | (%/g) |
| CE1 | fresh | 0.3 | 16.9 | 92.1 | 16 | 53.3 |
| CE2 | spent | 0.3 | 8.5 | 97.3 | 8 | 26.7 |
| CE3 | fresh | 0.75 | 35.2 | 88 | 31 | 41.3 |
| CE4 | spent | 0.6 | 16.7 | 98 | 16 | 26.7 |
| CE5 | fresh | 0.2 | 14 | 95 | 13.3 | 66.5 |
| CE6 | spent | 0.2 | 6.6 | 97 | 6.5 | 32.5 |
| IE1 | 200 C., 1 h, Air | 0.3 | 13.0 | 92.0 | 12 | 40.0 |
| IE2 | 200 C., 4 h, Air | 0.3 | 15.1 | 88.7 | 13 | 43.3 |
| IE3 | 250 C., 4 h, Air | 0.3 | 20.7 | 83.9 | 17 | 56.7 |
| IE4 | 300 C., 4 h, Air | 0.3 | 22.2 | 83.6 | 19 | 63.3 |
| IE5 | 350 C., 1 h, Air | 0.3 | 24.7 | 85.5 | 21 | 70.0 |
| IE6 | 350 C., 4 h, Air | 0.3 | 24.8 | 82.1 | 20 | 66.7 |
| IE7 | 600 C., 1 h, Air | 0.3 | 18.0 | 91.3 | 16 | 53.3 |
| IE8 | 350 C., 4 h, Air | 0.5 | 34.5 | 86 | 30 | 60.0 |
| IE9 | 400 C., 4 h, Air | 0.5 | 34.1 | 86 | 29 | 58.0 |
| IE10 | 450 C., 4 h, Air | 0.5 | 33.2 | 90 | 30 | 60.0 |
| IE11 | 300 C., 4 h, N₂ | 0.2 | 12.6 | 90 | 11.3 | 56.5 |
| IE12 | 350 C., 4 h, N₂ | 0.2 | 12.0 | 90 | 10.8 | 54.0 |
| IE13 | 400 C., 4 h, N₂ | 0.2 | 14.3 | 89 | 12.8 | 64.0 |
| IE14 | 300 C., 4 h, vacuum | 0.2 | 12.2 | 90 | 10.9 | 54.5 |
| IE15 | 350 C., 4 h, vacuum | 0.2 | 18.0 | 86 | 15.4 | 77.0 |
| IE16 | 400 C., 4 h, vacuum | 0.2 | 13.0 | 87 | 11.4 | 57.0 |

As can be seen from the normalized yields of Table 2, the catalyst loses about 50% of the activity after 1 to 1.5 hour of reaction (i.e., the spent catalysts of CE1, CE3 and CE5 (i.e. CE2 CE4 and CE6) produce half of the yield CE1, CE3 and CE5 do). The normalized yields of 1E1-1E6 and 1E8-16 surprisingly indicate that the catalysts regenerated from 200° C. to 425° C. provide yields comparable or even exceeding that of the fresh catalysts CE1 CE3 and CE5. Also unexpectedly discovered is that the normalized yield for the nitrogen and vacuum regenerated catalysts (IE11-IE16) are comparable or higher than that for the fresh samples (CE1, CE3 and CE5) despite oxygen not being present to oxidize and remove the fouling (i.e., the generally accepted theory of the prior art). As such, it has been unexpectedly be discovered that not only regeneration temperatures below 450° C. can provide superior monoalkyl ether production and comparable monoalkyl ether selectivity, but also that non-oxygen containing environments may also be utilized below 450° C.

1. A method, comprising the steps: (a) providing a metallosilicate catalyst that has been used to catalyze a chemical reaction; and (b) heating the metallosilicate catalyst to a temperature from 200° C. to 425° C. for a period of 0.5 hours to 5 hours.

2. The method of claim 1, wherein the step of heating the metallosilicate catalyst is performed in the absence of a liquid.

3. The method of claim 1, further comprising the step: catalyzing the chemical reaction between an olefin and an alcohol using the metallosilicate catalyst.

4. The method of claim 3, wherein the alcohol is selected from the group consisting of methanol, ethanol, monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof.

5. The method of claim 3, wherein the olefin comprises a C12-C14 alpha-olefin.

6. The method of claim 3, further comprising the step of: generating an (poly)alkylene glycol monoalkyl ether.

7. The method of claim 1, wherein the step of heating the metallosilicate catalyst lasts for a period of 1 to 4 hours.

8. The method of claim 7, wherein the step of heating the metallosilicate catalyst is performed at a pressure of less than 4000 Pa.

9. The method of claim 7, wherein the step of heating the metallosilicate catalyst is performed under an atmosphere comprising greater than 99 wt% nitrogen.

10. The method of claim 1, wherein the step of heating the metallosilicate 5 catalyst is performed at a temperature of from 250° C. to 400° C.

11. The method of claim 10, wherein the step of heating the metallosilicate catalyst is performed at a temperature of from 300° C. to 350° C.
